(19) United States
(12) Patent Application Publication
(10) Pub. No.: US 2011/0160754 A1
(43) Pub. Date: Jun. 30, 2011

(54) SPRING-LOADED ANASTOMOSIS DEVICE AND METHOD

(76) Inventor: Justin M. Crank, Maple Grove, MN (US)

(21) Appl. No.: 12/982,469

(22) Filed: Dec. 30, 2010

Related U.S. Application Data
(60) Provisional application No. 61/290,950, filed on Dec. 30, 2009.

(51) Int. Cl. A61B 17/11

(52) U.S. Cl. 606/153

(57) ABSTRACT

An anastomosis device and related methods for performing an anastomosis procedure, such as rejoining a urethra with a bladder following a radical prostatectomy. The engagement structures each have deployable tines for grasping the tissue proximate to the end of the biological lumen and the opening in the bodily organ. The anastomosis device also has a positioning apparatus for dynamically maintaining the engagement structures in proximity throughout a healing process such that tissue to be joined remains in approximation.

### PRIORITY CLAIM

The present application claims priority to U.S. Provisional Application Ser. No. 61/290,950, filed Dec. 30, 2009 and entitled “SPRING-LOADED ANASTOMOSIS DEVICE AND METHOD,” which is herein incorporated by reference in its entirety.

### FIELD OF THE DISCLOSURE

The present invention is generally directed to a surgical device and related methods for performing anastomosis procedures. Specifically, the present invention is directed to a spring-loaded surgical device for joining a severed biological lumen with an opening in a bodily organ during an anastomosis procedure.

### BACKGROUND OF DISCLOSURE

An anastomosis procedure typically involves connecting or reconnecting certain biological tissues such as biological lumen or organs. In most anastomosis procedures, a biological lumen comprising a blood vessel, intestinal, digestive or urinary tissue is severed and/or reconnected as part of the procedure. In other procedures, a severed biological lumen is connected or reconnected with a bodily organ. One representative example includes a radical prostatectomy procedure in which, a surgeon removes all or most of a patient's prostate. The procedure generally leaves a severed urethral stump and a severed bladder neck, which must be reconnected to so as to restore proper urinary functions.

An anastomosis device for performing an anastomosis procedure can involve an elongated device having two sets of mechanical tines for grasping the severed ends of the tissue to be reconnected and maneuvering the severed ends proximate to each other for suturing or other conventional means of connecting the severed ends together. A representative example of such an anastomosis device is described in U.S. Pat. No. 7,771,443, which is commercially available from the owner of the present application, American Medical Systems of Minnetonka, Minn., and for which the content is herein incorporated by reference in its entirety.

During an anastomosis procedure utilizing such an anastomosis device, a surgeon typically mechanically manipulates the two sets of mechanical tines to selectively engage and align the severed ends of tissue such that the biological lumen is restored so as to allow for the passage of biological fluid. The discrete diameter of the biological lumens increase the skill and technique required on the part of the surgeon to accurately align the severed ends of the lumen. Furthermore, anastomosis procedures performed in a minimally invasive manner involve advancing the anastomosis device through the biological lumen to reach the surgical site, which requires that the surgeon not only align the severed tissue ends but also make the determination that the severed tissue ends are proximately positioned either tactilely or visually through an optical fiber threaded through the biological lumen within the anastomosis device. In either case, considerable skill is often required on the part of the surgeon to properly attach the severed tissue ends together to fully restore the biological lumen without the benefit of verifying that the severed ends are properly joined through alternate views of the joined severed ends.

After the severed ends of the biological lumen are approximated and rejoined, the anastomosis device must often remain in place for a period of time to ensure the severed tissue ends do not separate before healing is complete. As many anastomosis devices are integrated into a catheter body for navigation through the biological lumen, a portion of the catheter body is often left in place within the biological lumen until the healing is complete which can cause discomfort for the patient.

While the anastomosis devices described above are used to effectively reconnect tissue during surgical procedures, it would be advantageous to improve upon their design so as to further assist the surgeon in aligning and rejoining the severed ends while minimizing the discomfort experienced by patients during the healing period.

### SUMMARY OF DISCLOSURE

The present invention is directed to anastomosis device adapted to rejoin a biological lumen with a bodily organ such as rejoining a urethra to a bladder following a radical prostatectomy. The anastomosis device is adapted operably engage the tissue proximate to the severed end of the biological lumen and the opening in the bodily organ and draw the biological lumen and bodily organ into approximation during a healing period. The anastomosis device can comprise a positioning apparatus for dynamically applying a force to maintain the biological lumen and bodily organ into contact or can comprise a guiding apparatus for assisting surgeons as they actively guide the biological lumen and bodily organ together.

An embodiment of an anastomosis device for use in a radical prostatectomy can generally comprise a urethra engagement portion, a bladder engagement portion and a positioning apparatus. The urethra engagement portion can comprise at least one deployable urethra tine adapted to be deployed when the urethra engagement portion is positioned proximate the severed end of the urethra. Similarly, the bladder engagement portion can comprise at least one deployable bladder tine adapted to be deployed when the urethra engagement portion is positioned proximate to the opening of the bladder. According to an embodiment of the present invention, the urethra engagement and bladder engagement portion can be positioned independently of each other to separately engage the tissue of the urethra and the bladder. Alternatively, the positioning apparatus can comprise a positioning spring adapted to dynamically apply a pulling or pushing force to one or both of the bladder engagement portion and the urethra engagement portion such that tissue remains approximated throughout a healing period. Alternatively, the positioning apparatus can comprise a positioning body adapted to push the urethra engagement portion toward the urethra engagement portion drawing the engaged tissue together for suturing. In this configuration, the positioning apparatus can further comprise a guide body to properly align the severed end of the biological lumen with the opening of the bodily organ.

According to an embodiment of the present invention, the anastomosis device can be positioned proximate to a severed end of the urethra and an opening of the bladder by an elongated catheter body navigable through the urethra. The elongated catheter body can define an inner lumen for receiving the anastomosis device. In this configuration, urethra tines and bladder tines remain un-deployed while the anastomosis device is positioned within the inner lumen of the elongated catheter body. Upon reaching the treatment site the anastomosis device is extended from the inner lumen to deploy the urethra and bladder tines so as to engage the tissue of the urethra and bladder. According to an embodiment of the present invention, the catheter body can further comprise a port for receiving the anastomosis device during navigation of the catheter body within the urethra.

In a representative method of the present invention, an anastomosis procedure following a radical prostatectomy can comprise advancing an anastomosis device through a severed urethra end and into a bladder opening. The method can further comprise deploying at least one bladder tine from a bladder engagement portion so as to grasp bladder tissue proximate the bladder opening. Next, the method can comprise deploying at least one urethra tine from a urethra engagement portion so as to grasp urethra tissue proximate the severed urethra end. Finally, the method can comprise applying force to at least one of the bladder engagement portion and the urethra engagement portion with a positioning spring to maintain the severed urethra end and the bladder opening in proximity throughout a healing period. In one representative embodiment, the step of applying force to at least one of the bladder engagement portion and the urethra engagement portion can comprise pulling the bladder engagement portion and the urethra engagement portion together with the positioning spring. In another representative embodiment, the step of applying force can comprise pushing the bladder engagement portion toward the urethra engagement portion together with the positioning spring. In yet another representative embodiment, the step of applying force can comprise pushing the urethra engagement portion toward the bladder engagement portion together with the positioning spring.

The above summary of the invention is not intended to describe each illustrated embodiment or every implementation of the present invention. The Figures and the Detailed Description that follow more particularly exemplify these embodiments.

### BRIEF DESCRIPTION OF FIGURES

The invention can be more completely understood in consideration of the following detailed description of various embodiments of the invention in connection with the accompanying drawings, in which:

FIG. 1 is a representative cross-sectional view of a male urinary tract.

FIG. 2 is a perspective view of a minimally invasive surgical device for positioning an anastomosis device according to an embodiment of the present invention.

FIG. 3 is a cross-sectional side view of an anastomosis device according to an embodiment of the present invention.

FIG. 4 is a representative perspective view of the anastomosis device of FIG. 3 maintaining a severed urethra end in approximation with an bladder opening.

FIG. 5 is a cross-sectional side view of an anastomosis device according to an embodiment of the present invention.

FIG. 6A is a front view of a bladder tine array of an anastomosis device according to an embodiment of the present invention.

FIG. 6B is a side view of the bladder tine array depicted in FIG. 6A.

FIG. 7 is a cross-sectional side view of an anastomosis device according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF FIGURES

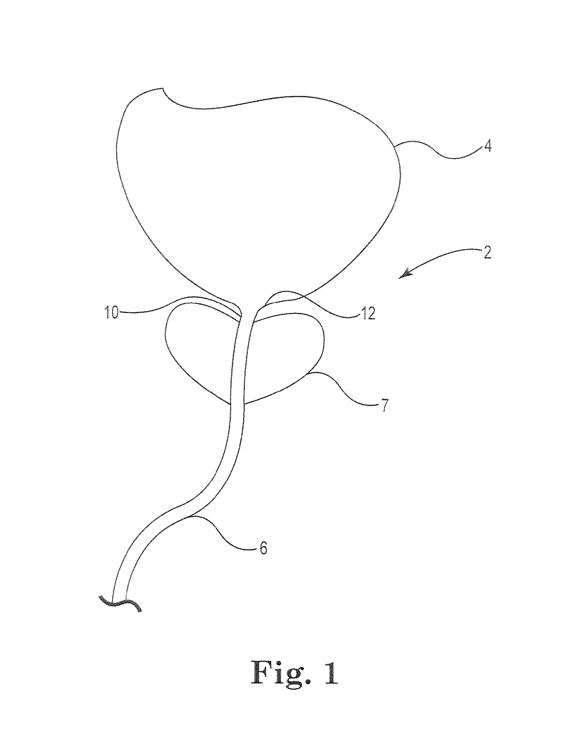

As shown in FIG. 1, a typical male urinary tract 2 comprises a bladder 4 for storing a quantity of urine and a urethra 6 fluidly connected to the bladder 4. In males, a prostate gland 7, which is positioned around the urethra 6 proximate to the junction of the urethra 6 and the bladder 4, can become inflamed or diseased requiring surgical removal of at least a portion of the prostate 7. In a radical prostatectomy procedure, the prostate 7 and typically a portion of the urethra 6 proximate to the prostate 7 is surgically removed. The radical prostatectomy procedure results in the urethra 6 being severed from the bladder 4 so as to allow removal of the prostate 7 and/or a portion of the urethra 6. As a result, an anastomosis procedure is typically performed following the prostatectomy to rejoin a severed urethra end 10 with a bladder opening 12 formed by severing the urethra 6 so as to restore proper urinary function for the patient.

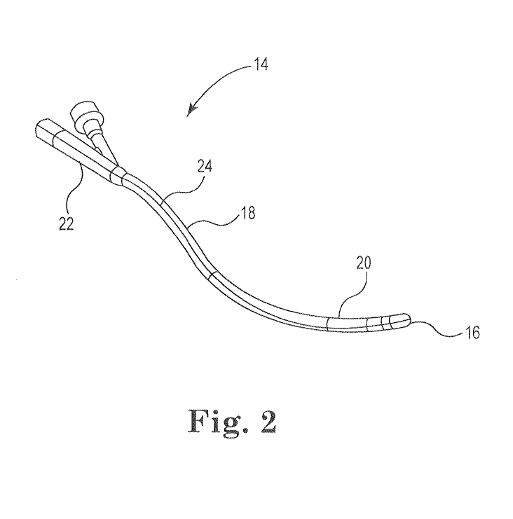

As shown in FIG. 2, a minimally invasive surgical device 14 for performing anastomosis procedures, according to an embodiment of the present invention, can comprise an anastomosis device 16 and an elongated catheter body 18. The elongated catheter body 18 comprises an open distal end 20 that is inserted into the patient's body and an open proximal end 22 that remains outside the patient's body. The elongated catheter body 18 also defines a lumen 24 extending between the distal end 20 and the proximal end 22 and adapted to receive the anastomosis device 16.

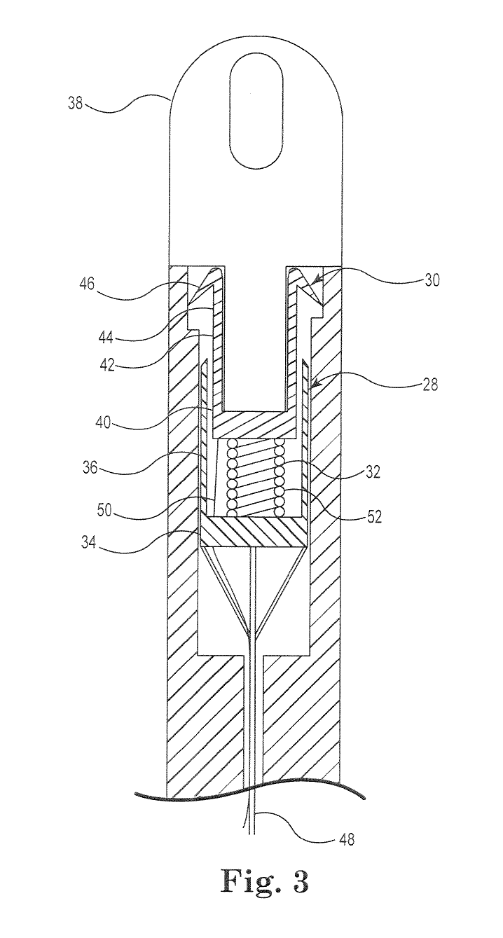

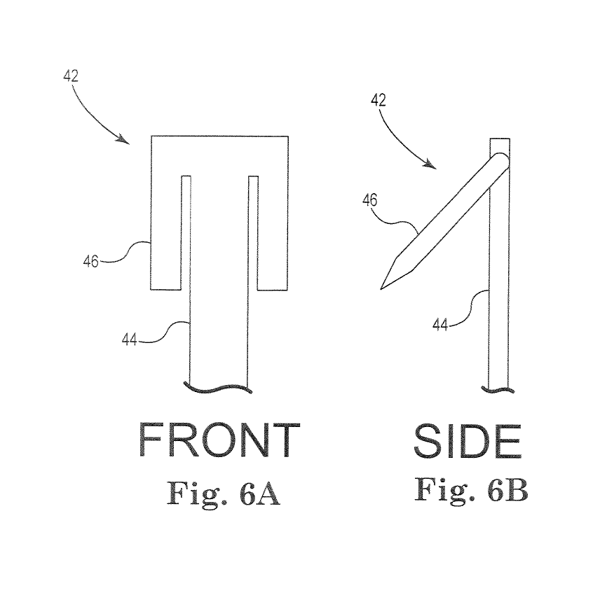

In one representative embodiment, anastomosis device 16 can comprise a urethra engagement portion 28, a bladder engagement portion 30 and a positioning apparatus 32 as shown in FIG. 3. The urethra engagement portion 28 can further comprise a urethra base 34 and at least one urethra tine 36. The bladder engagement portion 30 can further comprise a catheter tip 38, a bladder base 40 and at least one bladder tine array 42. Each bladder tine array 42 can further comprise a shaft 44 and at least one bladder tine 46 as shown in FIGS. 6A and 6B.

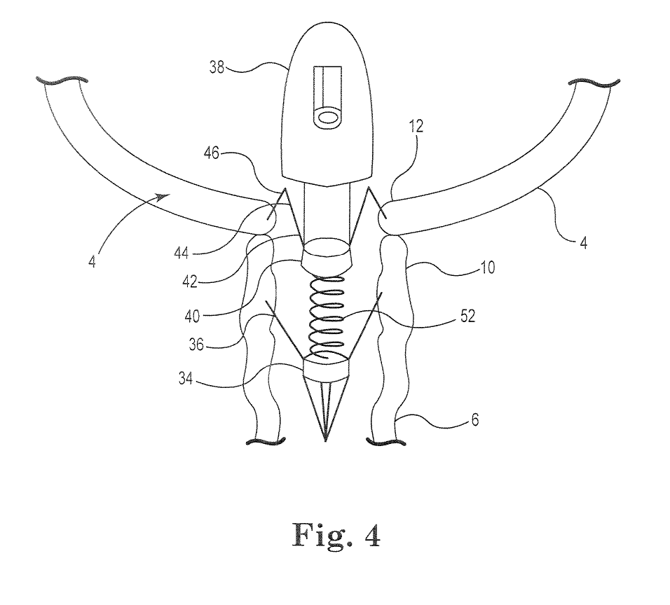

Urethra engagement portion 28 is generally adapted to engage the walls of the urethra 6 proximate to the severed urethra end 10 with the urethra tines 36. The urethra tines 36 are movable between a retracted position in which the urethra tines 36 are generally parallel with the longitudinal axis a-a of the catheter body 18 for transport to the surgical site within the catheter body 18 as shown in FIG. 3 and a deployed position in which the urethra tines 36 extend outwardly from the longitudinal axis a-a of the catheter body 18 for engaging the walls of the urethra 6 as shown in FIG. 4. According to an embodiment of the present invention, the urethra tines 36 can be biased toward the deployed position such the urethra tines 36 passively deploy as soon as the anastomosis device 16 is deployed from the lumen 24 of the catheter body 18. Alternatively, the urethra engagement portion 26 can further comprise a deployment wire 48 extending through the catheter body 18 that can be manipulated to manually deploy the urethra tines 36.

In a similar manner, bladder engagement portion 30 is generally adapted to engage the bladder 6 proximate the bladder opening 12 with the bladder tines 46. As with the urethra tines 36, the bladder tines 46 are movable between a retracted position in which the bladder tines 46 are generally parallel with the longitudinal axis a-a of the catheter body 18 for transport to the surgical site within the catheter body 18 as shown in FIG. 3 and a deployed position in which the bladder tines 46 extend outwardly from the longitudinal axis a-a of the catheter body 18 for engaging the tissue proximate the bladder opening 12 as shown in FIG. 4. According to an embodiment of the present invention, bladder tines 46 can be biased toward the deployed position such the bladder tines 46 passively deploy as soon as the anastomosis device 16 is deployed from the lumen 24 of the catheter body 18. Alternatively, the bladder engagement portion 30 can further comprise a deployment wire 50 extending through the catheter body 18 that can be manipulated to manually deploy the bladder tines 46.

As shown in FIG. 4, the urethra tines 36 are generally pointed toward the distal end 20 of the catheter body 18 when deployed, while the bladder tines 46 are generally pointed toward the proximal end 22 of the catheter body 18. As described in detail below, the opposing directions of the urethra tines 36 and the bladder tines 46 allow a pulling or pushing force to be applied to the urethra 6 and the bladder 4 to draw the severed urethra end 10 into proximity with the bladder opening 12. According to an embodiment of the present invention, the urethra tines 36 and the bladder tines 46 can comprise stainless steel, nickel titanium, titanium, MP35N Nickel-Cobalt Alloy, or other suitable biocompatible metal.

As shown in FIGS. 3 and 4, the urethra engagement portion 28 is movable relative to the bladder engagement portion 30, wherein the positioning apparatus 32 governs the relative distance between the urethra engagement portion 28 and the bladder engagement portion 30. As shown in FIG. 3, according to an embodiment of the present invention, the positioning apparatus 32 can comprise a positioning spring 52 operably linking the base 34 of the urethra engagement portion 28 to the base 40 of the bladder engagement portion 30. The positioning spring 52 is adapted to apply a pulling force to the urethra engagement portion 28 and the bladder engagement portion 30 to draw the urethra engagement portion 28 and the bladder engagement portion 30 together. Positioning spring 52 responds dynamically to tissue stress during the healing period such that the severed urethra end 10 and the bladder opening 12 remain approximated throughout the healing period.

To perform an anastomosis procedure, the distal end 20 of the catheter body 18 is navigated through the urethra 6, out the severed urethra end 10 and into the bladder opening 12. The anastomosis device 16 is extended partly out of the proximate end 20 of the catheter body 18 such that the bladder tines 46 extend outwardly from the longitudinal axis a-a of the catheter body 18 wherein the bladder tines 46 engage tissue with the bladder 4 proximate the bladder opening 12. While grasping the bladder 4 with bladder tines 46, the urethra tines 36 are extended outwardly from the longitudinal axis a-a of the catheter body 18 to engage the urethra 6 proximate the severed urethra end 10. Positioning spring 52 applies a pulling force between the urethra engagement portion 28 and the bladder engagement portion 30 to continually pull the urethra 6 into contact with the bladder 4. Various tissue stresses can be imparted during the healing period such as, for example, due to movement of the patient, whereby the amount of pulling force imparted by positioning spring 52 dynamically adjusts to maintain approximation of the bladder opening 12 and severed urethra end 10. In some embodiments, anastomosis device 16 can be separated from the catheter body 18, wherein the anastomosis device 16 can be left in place for a predetermined period of time until the urethra 6 and the bladder 4 have healed together, while the catheter body 18 is removed from the patient's body to minimize discomfort to the patient.

A method for performing an anastomosis procedure can comprise providing the anastomosis device 16 having a urethra engagement portion 30 with deployable urethra tines 36 for engaging tissue proximate the severed urethra end 10 and a bladder engagement portion 28 having deployment bladder tines 46 for engaging tissue proximate to the bladder opening 12. The method further comprises advancing the anastomosis device 16 through the urethra 6 and into the bladder 4 such that the anastomosis device is proximate to the tissue around the bladder opening 12. The method can further comprise deploying bladder tines 46 to grasp tissue proximate the bladder opening 12. The urethra engagement portion 30 can then be independently positioned proximate the severed urethra end 10, wherein the urethra tines 36 can be deployed to grasp the urethra 6 proximate the severed urethra end 10. The method can further comprise applying force with the position apparatus 32 to maintain approximation of the severed urethra end 10 and the bladder opening 12 wherein the amount of force applied by positioning apparatus 32 dynamically responds to tissue stresses. The severed urethra end 10 and bladder opening 12 are maintained in approximation throughout the healing period.

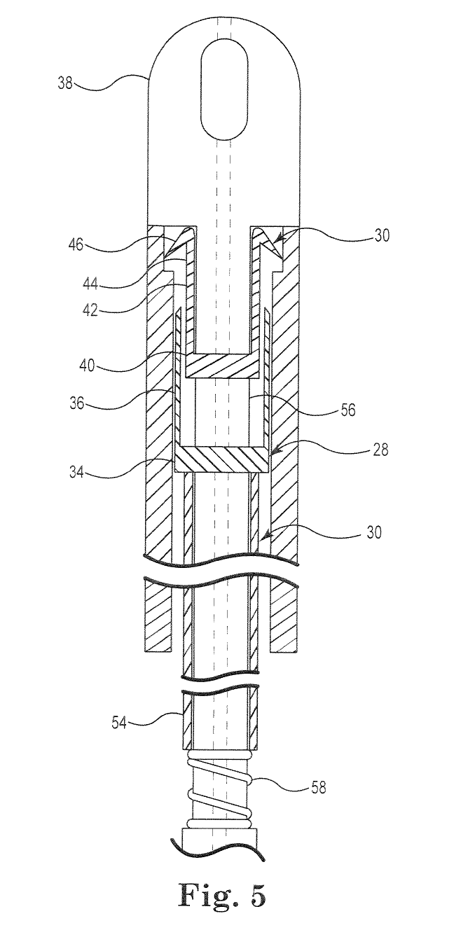

Referring not to FIG. 5, another representative embodiment of the anastomosis device 16 can comprise locating, the positioning apparatus 32 between the proximal end 22 and the urethra engagement portion 28. Anastomosis device 16 can comprise an elongated positioning body 54 extending the length of the catheter body 18. The positioning body 54 is affixed to the base 34 of the urethra engagement portion 28 and adapted to apply a pushing force to the urethra engagement portion 28 to move the urethra engagement portion 28 toward the bladder engagement portion 30. According to an embodiment of the present invention, the positioning apparatus 32 can further comprise a guide 56 for maintaining the alignment of the urethra engagement portion 28 with bladder engagement portion 30 as the urethra engagement portion 28 is pushed toward the bladder engagement portion 30. As discussed previously, positioning apparatus 32 can further comprise a positioning spring 58 for biasing the urethra engagement portion 28 toward the bladder engagement portion 30 by applying a pushing force on the positioning body 54. Once again, positioning spring 58 responds dynamically to tissue stress during the healing period by pushing the severed urethra end 10 against the bladder opening 12 such that they remain approximated throughout the healing period.

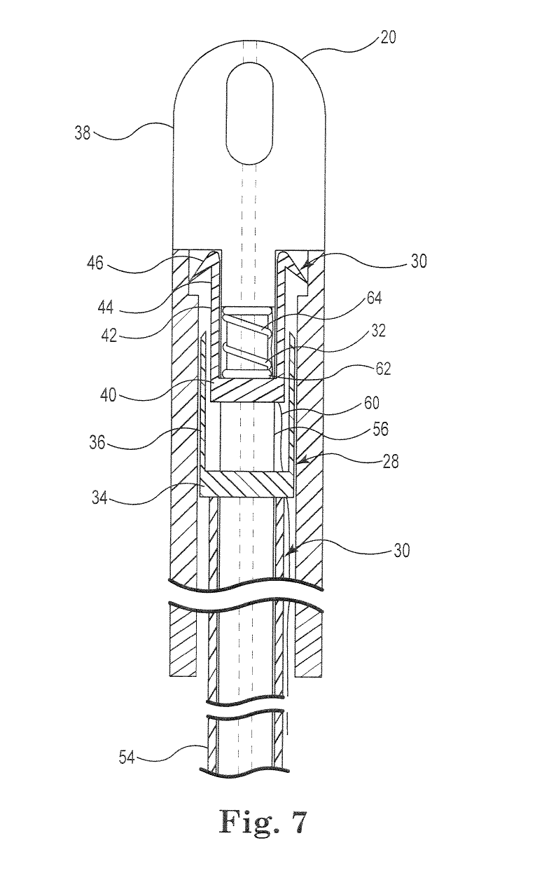

In another alternative embodiment as shown in FIG. 7, the anastomosis device 16 can comprise locating the positioning apparatus 32 between the distal end 20 and the bladder engagement portion 30. Anastomosis device 16 can comprise a positioning wire 60 extending the length of the catheter body 18 and attaching to a tip base 62. The positioning apparatus 32 is positioned between the tip base 62 and the bladder base 40. Positioning wire 60 allows for tip base 62 to be pulled toward the bladder base 40, thereby compressing the positioning apparatus 32 such that a pushing force is applied to the bladder engagement portion 30 to move the bladder engagement portion 30 toward the urethra engagement portion 28. As discussed previously, positioning apparatus 32 can further comprise a positioning spring 64 for biasing the bladder engagement portion 30 toward the urethra engagement portion 28 wherein positioning spring 64 responds dynamically to tissue stress during the healing period.

In operation, the anastomosis device 16 is stored within the lumen 24 of the catheter body 18 while the surgical device 14 is navigated through the urethra 6 until the proximate end 20 of the catheter body 18 is proximate to the surgical site at which point the anastomosis device 14 is deployed from the lumen 24 to rejoin the severed end 10 of the urethra 6 with the opening 12 of the bladder 4. According to an embodiment of the present invention, the elongated catheter body 18 can further comprise a receiving port 26 proximate to the proximate end 20 of the catheter body 18 for receiving at least a portion of the anastomosis device 16 during navigation of the surgical device 14 through the urethra 6.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

What is claimed:

1. An anastomosis device, comprising:
a distal engagement portion having at least one distal tine for selectively engaging a distalmost tissue structure;
a proximal engagement portion having at least one proximal tine for selectively engaging a proximalmost tissue structure; and
a positioning apparatus operably coupled to at least one of the distal engagement portion or the proximal engagement portion, the positioning apparatus dynamically responding to tissue stress to maintain approximation of the distalmost tissue structure and the proximalmost tissue structure.

2. The anastomosis device of claim 1, wherein the positioning apparatus comprises a positioning spring.

3. The anastomosis device of claim 2, wherein the positioning spring is operably coupled between the distal engagement portion and the proximal engagement portion such that the positioning spring pulls the distal engagement portion and the proximal engagement portion into proximity.

4. The anastomosis device of claim 2, wherein the positioning spring is operably coupled between the distal engagement portion and a distal tip such that the positioning spring pushes the distal engagement portion toward the proximal engagement portion.

5. The anastomosis device of claim 2, wherein the positioning spring is operably coupled between the proximal engagement portion and a proximal wall such that the positioning spring pushes the proximal engagement portion toward the distal engagement portion.

6. The anastomosis device of claim 2, wherein the at least one distal tine is passively deployed from the distal engagement portion.

7. The anastomosis device of 2, wherein the at least one proximal tine is passively deployed from the proximal engagement portion.

8. An anastomosis device for use in a radical prostatectomy, comprising:
a bladder engagement portion having at least one bladder tine for selectively engaging a bladder opening;
a urethra engagement portion having at least one urethra tine for selectively engaging a severed urethra end; and
a positioning apparatus operably coupled to at least one of the bladder engagement portion or the urethra engagement portion, the positioning apparatus dynamically responding to tissue stress to maintain approximation of the bladder engagement portion and the urethra engagement portion throughout a healing period.

9. The anastomosis device of claim 8, wherein the positioning apparatus comprises a positioning spring.

10. The anastomosis device of claim 9, wherein the positioning spring is operably coupled between the bladder engagement portion and the urethra engagement portion such that the positioning spring pulls the bladder engagement portion and the urethra engagement portion into proximity.

11. The anastomosis device of claim 9, wherein the positioning spring is operably coupled between the bladder engagement portion and a distal tip such that the positioning spring pushes the bladder engagement portion toward the urethra engagement portion.

12. The anastomosis device of claim 9, wherein the positioning spring is operably coupled between the urethra engagement portion and a proximal wall such that the positioning spring pushes the urethra engagement portion toward the bladder engagement portion.

13. The anastomosis device of claim 9, wherein the at least one bladder distal tine is passively deployed from the bladder engagement portion.

14. The anastomosis device of 9, wherein the at least one urethra tine is passively deployed from the urethra engagement portion.

15. A method for performing anastomosis following a radical prostatectomy, comprising:
advancing an anastomosis device through a severed urethra end and into a bladder opening;
deploying at least one bladder tine from a bladder engagement portion so as to grasp bladder tissue proximate the bladder opening;
deploying at least one urethra tine from a urethra engagement portion so as to grasp urethra tissue proximate the severed urethra end; and
applying force to at least one of the bladder engagement portion and the urethra engagement portion with a positioning spring to maintain the severed urethra end and the bladder opening in proximity throughout a healing period.

16. The method of claim 16, wherein applying force to at least one of the bladder engagement portion and the urethra engagement portion comprises pulling the bladder engagement portion and the urethra engagement portion together with the positioning spring.

17. The method of claim 16, wherein applying force to at least one of the bladder engagement portion and the urethra engagement portion comprises pushing the bladder engagement portion toward the urethra engagement portion together with the positioning spring.

18. The method of claim 16, wherein applying force to at least one of the bladder engagement portion and the urethra engagement portion comprises pushing the urethra engagement portion toward the bladder engagement portion together with the positioning spring.
